(19) United States
(12) Patent Application Publication
(10) Pub. No.: US 2010/0015601 A1
(43) Pub. Date: Jan. 21, 2010

(54) BIOLOGICAL CONFIRMATION AND DETECTION SYSTEM

(75) Inventors: James Gilmore, Del Mar, CA (US); James Meegan, Woodbine, MD (US)

Correspondence Address: LIFE TECHNOLOGIES CORPORATION;C/O INTELLEVATE, P.O. BOX 52050, MINNEAPOLIS, MN, 55402, US

(73) Assignee: INVITROGEN CORPORATION, Carlsbad (CA)

(21) Appl. No.: 11/719,703

(22) PCT Filed: Nov. 23, 2005
(86) PCT No.: PCT/US2005/042894

§ 371 Date: Sep. 29, 2009

Related U.S. Application Data
(60) Provisional application No. 60/630,634, filed on Nov. 24, 2004.

(51) Int. Cl. C12Q 1/68; C12M 1/00; C40B 60/12; C40B 30/00

(52) U.S. Cl. 435/6; 435/287.2; 506/39; 506/7

(57) ABSTRACT

The present disclosure describes a method and apparatus for collecting samples of particles from air or other gases at one or more monitored locations, and identifying in real-time whether biological agents, such as bacterial or viral pathogens, are present in the samples. The apparatus preferably uses a liquid-assisted collector to collect the sample of particles, which are suspended in a liquid that contains dyes that detect the presence of nucleic acids. An integrated detector with a light source and a light detector detects whether there is a change in the fluorescence of the liquid, which indicates the presence of a biological agent in the sample.

### STATEMENT REGARDING FEDERALLY SPONSORED RESEARCH OR DEVELOPMENT

Not applicable.

### REFERENCE TO A “MICROFICHE APPENDIX”

Not applicable.

### BACKGROUND OF THE INVENTION

1. Field of the Invention

This invention is directed to collection and detection systems for the real-time detection of increases in the presence of biohazards or other harmful emissions, such as a biological pathogen.

2. Description of Related Art

Collector systems such as cyclones and impingers are used to extract trace contaminants from the air or other gas, thereby providing samples which may be monitored for harmful emissions or biohazards. For example, wet cyclone concentrators disclosed in U.S. Pat. Nos. 4,117,714, 5,011,517, 5,679,580, 5,855,652, 5,861,316, 5,988,603, 6,468,330, and 6,565,811 (each incorporated herein by reference) collect samples of particles from air or other gas, which may then be analyzed for the presence of a contaminant such as a biohazard. Liquid-assisted cyclone concentrators work by introducing a contaminant laden gas into a substantially cylindrical or conical shaped chamber so as to induce a swirling movement that brings the gas in intimate contact with a scrubbing liquid such as water along the walls of the chamber. Due, in part, to the centripetal motion of the contaminants as the gas swirls about the walls of the container, this contact allows impurities in the air to be extracted into the water, which may be withdrawn and analyzed. Unfortunately, collectors in current use do not possess detector characteristics. Instead, the detector that analyzes the collected samples is typically found in a downstream component, which is often not integrated with the collector, or is remote from the collector. Therefore, there is a need to convert existing collectors to broad range point detector systems. Preferably, the system would be able to monitor and signal in real-time any sudden increase in a potential biohazard or other harmful emissions, such as a biological pathogen or toxin. In addition, the system further would be able to identify the particular biohazard or other harmful emission, thereby alerting its users of the biological threat identified.

### BRIEF SUMMARY OF THE INVENTION

The present disclosure describes the conversion of existing collectors into broad range point detectors, such as biological confirmation and detection systems (BioCADS). BioCADS combines an automated collector apparatus, for example a collector apparatus that can specifically collect biohazards such as bacterial or viral pathogens, with a means for detecting potential biohazards in real-time. A primary advantage of this system is that it is able to detect an increase in the presence of a potential biohazard at or near the time of collection. The detection of a sudden increase or spike in the presence of a potential biohazard, such as bacterial spores, for example from contaminated letters being sorted in a mail system or in public or private transportation systems, may provide an early warning of a biological threat, such as the release of a bacterial pathogen. In preferred embodiments, BioCADs is able to: 1) determine the number of bacteria or virus present in a monitored area in real time; 2) determine whether the bacteria are Gram-negative or Gram-positive; and/or 3) distinguish whether the bacteria identified are live or dead.

BioCADS of the present disclosure utilize different dyes to allow the identification of potential biohazards, such as bacterial or viral pathogens, while particles are collected in real-time. Sensitivity of BioCADS is critical, since this may be the difference between detecting and missing a lethal threat.

In certain embodiments, BioCADS includes means for particle collection, for example a liquid-assisted concentrator, and means for detecting the presence of biohazards, such as bacterial or viral pathogens, as they are extracted into the liquid of the concentrator. In other embodiments, BioCADS includes means for particle collection and pre-separation using a collection hood or other means capable of collecting emitted particulates from air or other gas, such as wet or dry cyclone passive filtration systems; means for identifying increases in the presence of biohazards such as bacterial or viral pathogens as they are extracted from air or other gas; continuous particle collection into a liquid sample; and/or means for transferring the liquid sample to a microfluidic microarray chip or flow cytometer that allows identification of the potential biohazard. BioCADS may also allow for automatic retesting upon various error conditions; automatic confirmation testing upon preliminary positive results; automated fluid transfer to archive containers at the completion of analysis; and an automated notification/reporting system to alert designated personnel/organizations upon the occurrence of selected events. BioCADS may further include an optical trigger device, for example, to identify particle concentration spikes that occur in real-time. Preferably, the optical trigger device operates in parallel with the continuous collection process.

BioCADS is particularly useful in certain environments, such as the U.S. Postal Service (USPS) or private and public transportation systems, because it is able to detect the presence of potentially toxic biological agents in a mail processing facility or transport system. BioCADS would notify the appropriate personnel in real-time of any sudden spike in the presence of a potential biohazard, so that appropriate actions may be taken quickly to contain the potential threat, thereby preventing dispersion into or contamination of the general public.

The present disclosure describes a system for detecting a biological agent, comprising a collector/concentrator apparatus for collecting a sample of particles from a gas at a monitored location, wherein the sample of particles is suspended in a liquid comprising one or more dyes (e.g., dyes that detect the presence of nucleic acids); and an integrated detector (e.g., that has a light source and a light detector), wherein the integrated detector can detect a change in the fluorescence of the liquid, wherein increased fluorescence of the liquid indicates the presence of a biological agent in the sample. The biological agent detected may be a biohazardous agent, such as a bacterial pathogen, a viral pathogen, or other toxin. In preferred embodiments, the collector/concentrator apparatus is a liquid-assisted collector, for example a SpinCon® apparatus. The collector/concentrator apparatus may operate continuously to collect samples of particles, or may only be directed to collect particles upon a threshold event, for example, detection by a particle counter of a certain number of particles per second in a certain size range passing by an air sample point. The system for detecting a biological agent disclosed herein may also have a dry-cyclone particle separator, a particle counter, and/or a bio-identifier apparatus (e.g., a polymerase chain reaction (PCR) biological agent identifier or a microfluidic microarray biochip).

The integrated detector may have a light source and a light detector, with the light source preferably being a lamp, for example a White Arc lamp, and/or a light detector which is preferably an optical detector, a photomultiplier tube, or a fluorometer. The integrated detector is not limited to a single light source and light detector, and may have a plurality of one or both of these components. The light source of the integrated detector may be place inside or outside the collector/concentrator apparatus. Similarly, the light detector may be place inside or outside the collector/concentrator apparatus. The light source and the light detector may also be adjacent to the collector/concentrator apparatus. Preferably the light from the light source will traverse at least one wall of the collector/concentrator apparatus, for example two or more walls, etc. The integrated detector may also have a bandpass filter.

Other embodiments of the present disclosure are methods of detecting a biological agent by collecting a sample of particles from a gas at a monitored location using a liquid-assisted collector, for example wherein the liquid-assisted collector comprises a liquid with one or more dyes (e.g., dyes that detect the presence of nucleic acids), exposing the sample to a light source; and detecting the fluorescence of the liquid in the sample with a light detector, wherein increased fluorescence of the liquid in the sample indicates the presence of a biological agent. The biological agent detected may be a biohazardous agent, such as a bacterial pathogen, a viral pathogen, or other toxin. In preferred embodiments, the liquid-assisted collector is a SpinCon® apparatus. The liquid-assisted may operate continuously to collect samples of particles, or may only be directed to collect particles upon a threshold event, for example, detection by a particle counter of a certain number of particles per second in a certain size range passing by an air sample point. The methods of detecting a biological agent disclosed herein may use a dry-cyclone particle separator, a particle counter, and/or a bio-identifier apparatus (e.g., a polymerase chain reaction (PCR) biological agent identifier or a microfluidic microarray biochip), to facilitate detection and/or identification of the biological agent.

In certain embodiments of the present disclosure, dyes that detect the presence of nucleic acids may be selected from the group consisting of PICOGREEN®, calcein AM, ethidium homodimer, SYTO® 9, propidium iodide, and hexidium iodide. In the above methods, detection of increased fluorescence may indicate the presence of a live bacterial pathogen or the presence of a dead bacterial pathogen. A detection of increased fluorescence may also indicate the presence of a Gram-positive or Gram-negative bacterial pathogen.

The light source used in the methods disclosed herein may be a lamp, for example a White Arc lamp, while the light detector used may be an optical detector, a photomultiplier tube, or a fluorometer. A plurality of light sources and/or light detectors may also be used in these methods.

### BRIEF DESCRIPTION OF THE SEVERAL VIEWS OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a system block diagram illustrative of a bio-detection system in accordance with an embodiment of the invention.

FIG. 2 is a system block diagram illustrative of the apparatus located in the monitor unit of FIG. 1.

FIG. 3 is a schematic representation of a liquid-assisted collector incorporating in-line detection apparatus in accordance with one embodiment of the invention.

FIG. 4(a) and FIG. 4(b) are flow diagrams illustrating the steps performed by a processor that controls the fluid level in a liquid-assisted collector.

FIG. 5(a) is a flow diagram illustrating the steps performed by a processor that controls the in-line detection apparatus in accordance with one embodiment of the invention.

FIG. 5(b) is a flow chart illustrative of the operation of a bio-detection system in accordance with an embodiment of the invention.

FIG. 6 and FIG. 7 are schematic representations of a liquid-assisted collector incorporating an in-line detection apparatus in accordance with alternative embodiments of the presently disclosed system.

FIG. 8 is a schematic representation of a bio-identifier apparatus in accordance with an embodiment of the invention.

### DETAILED DESCRIPTION OF THE INVENTION

### System Overview

Referring now to the various drawings figures where like reference numerals refer to like components throughout, shown throughout is a biological confirmation and detection system (BioCADS).

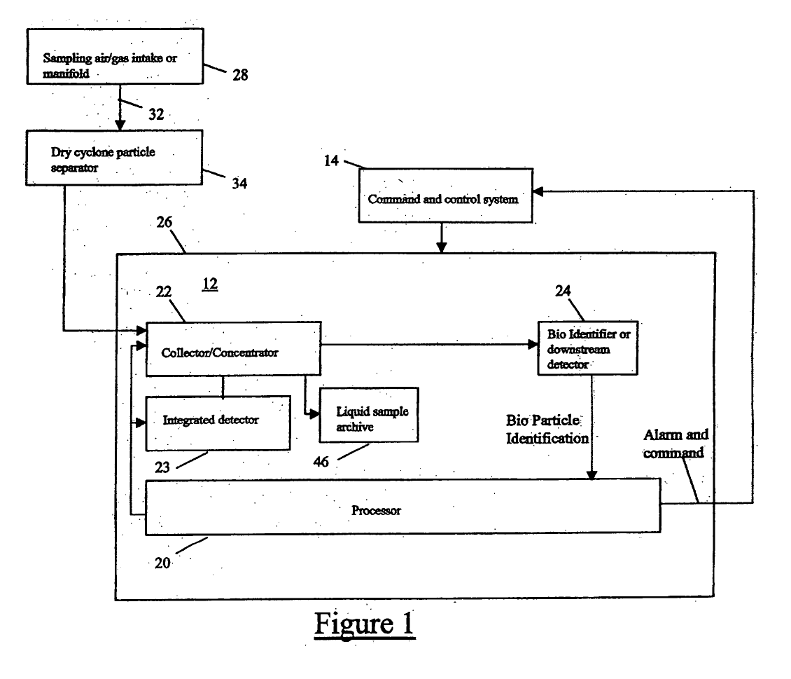

FIG. 1 illustrates a BioCADS 10 with a single monitor unit 12; however, more than one monitor unit can be employed depending on the needs of the particular facility. In either case, one or a plurality of the monitoring units 12 may be under the control of a central site command and control system 14 (FIG. 1). The monitor unit 12 can be coupled to the site command and control system 14 either by way of a hardwired network or an RF link, as desired. Each monitor unit 12 includes two major sub-systems under the control of a machine control processor 20, namely: a collector/concentrator 22 and an integrated detector 23 located in a cabinet shown by reference numeral 26. The collector assembly preferably also includes an integrated detector 23 (FIG. 1), operable to detect one or more biological agents inside the liquid assisted collector/concentrator assembly 22. Preferably the collector/concentrator 22 is a fluidics transfer sub-system, for example a liquid assisted concentrator. An optional bio-identifier sub-system 24 is also depicted.

In addition to the monitor unit 12, the subject BioCADS 10 as shown in FIG. 1 includes an air intake or manifold system 28 for sampling the air around one or more specific points. Sampling can be performed by drawing air into the air intake, thereby capturing the particles at the specific intake point or points.

The air from the intake or manifold 28 can, when desired, be continuously monitored by an optional particle counter, not shown, which determines the number of particles per second in a number of size ranges passing by the air sample point. Such an option would provide a historical record of particle count that may be useful in identifying the point or points at which the contaminated sample was taken and the approximate time at which the sample was taken in the event the monitor unit 12 detects a biological agent. If a spike is detected in the counted particles with characteristics that match a potential biohazard, the system can also use this event to automatically trigger a sample analysis process described hereinafter. Particle characteristics evaluated include but are not limited to count, size, shape, and fluorescence signature, among others. It is also possible to use a mass spectrometer, not shown, as a trigger. As noted, BioCADS in accordance with the present disclosure may operate with or without a particle counter at the air intake 28.

After the particles are captured, they preferably are sent via a hose 32 through a dry cyclone 34, that utilizes the particle aerodynamic size to separate out larger particles from those that are in the inhalable size range, and therefore pose the highest threat to human health. This cleans up the aerosol sample, and prevents large dust and fibrous particles from clogging the downstream equipment and interfering with the biodetection process. The large particles are captured in a container, not shown, and disposed of. Preferably, no filter media that can become clogged with dust is utilized.

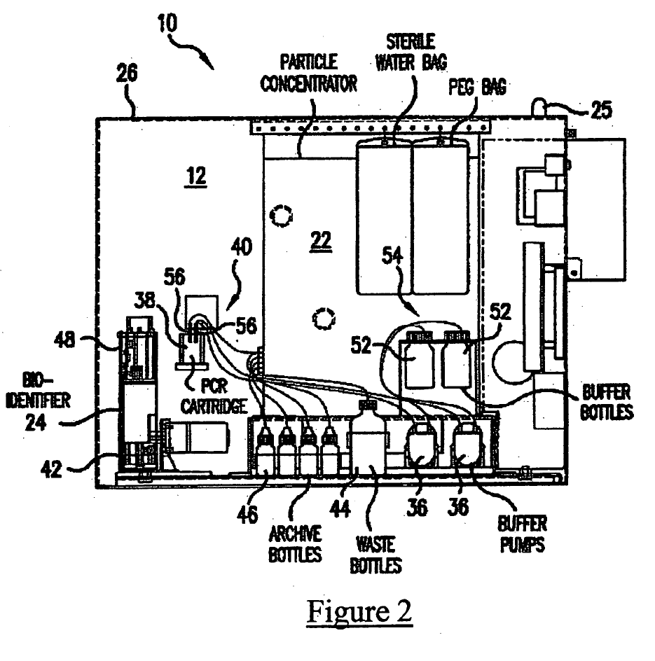

Referring now to FIG. 2, a liquid assisted collector/concentrator assembly 22 is preferably a SpinCon® system, which constantly draws an air sample from the intake or manifold 28 and the dry cyclone particle separator 34 and impinges the sample into a small amount of liquid located in a glass or plastic collector, not shown. If the integrated detector 23 of the SpinCon® system detects an increase in the presence of a potential biohazard, the machine control processor 20 (FIG. 1) may direct a small amount of collected sample out of the collector to a reservoir where it is analyzed by a bio-identifier apparatus 24. As illustrated in FIG. 2, this bio-identifier apparatus 24 may be a polymerase chain reaction (PCR) cartridge 38 at a fill station 40. (See U.S. Publ. Nos. 2004/0063197 and 2004/0063198, each incorporated herein by reference).

When the machine control processor 20 directs the bio-identifier apparatus 24 to analyze a sample, the analysis may be either positive for a particular biohazard, or non-determinate, indicating that certain internal controls did not perform correctly, and further analyses may be performed using additional fractions of the original sample. At the completion of the analysis, the remaining sample may be transferred from the reservoir into a waste bottle 44, or to archive bottles 46 for later laboratory confirmatory analysis and retention as evidence. The system can optionally individually archive all samples or only those that generate a positive test result. The bio-identifier apparatus 24 may be controlled by the central site command and control system 14 (FIG. 1).

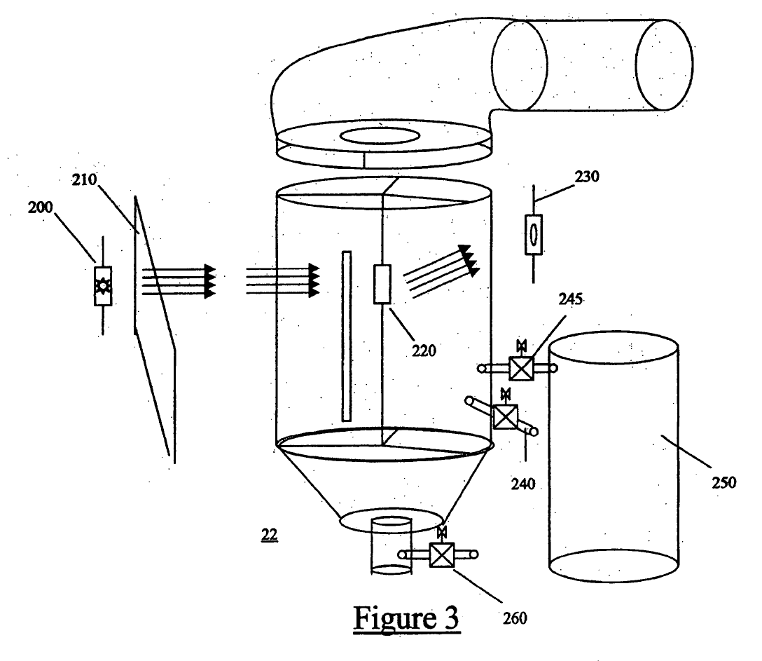

In another embodiment, illustrated by FIG. 3, the integrated detector 23 includes a lamp 200 and bandpass filter 210, positioned to direct light into the liquid assisted collector/concentrator assembly 22. The lamp used in BioCADS may be, for example, a white arc lamp, a light emitting diode (LED), an incandescent light, or alternatively a laser source. The light passes through the glass or plastic collector where it preferably interacts with a dye that has been added to the liquid in the collector in order to bind to a potential biohazard, such as a biological agent. As the light passes through the collector, it may be diverted by an optional reflective or transmissive optical device 220 thereby passing to a detector 230 which converts light into an electric signal. This detector 230 may constitute a photomultiplier tube, semiconductor detector, or other optical detectors including visual inspection, cameras and film or other imaging equipment, or instruments such as fluorometers, plate readers, laser scanners, microscopes, or flow cytometers.

The dye may be added to the glass or plastic collector through its own port 240, or it may be mixed with the collection liquid in the collector source reservoir 250 before the liquid is injected into the collector. The port 240 is shown for illustrative purposes. In the event that multiple dyes are used in the system, it would be possible to inject all of the dyes through a common port, or to provide a plurality of dye inlet ports to provide dye to the collector. The use of a plurality of inlet ports would allow different dyes to be injected at different rates into the collector/concentrator assembly 22 to optimize detection of potential biohazards. The amount and rate of dye injected into the system may be controlled by processor 22 based on feedback from an optional particle counter, for example based on the size or rate of particles being directed to the concentrator/collector assembly 22. A plurality of different dyes could also be dissolved in the collection liquid stored in the collection source reservoir 250, and injected into the collector as collection liquid is added.

The level of liquid and dye in the collector may be controlled through the use of a computer controller (not shown), which may be the processor 20 of FIG. 1. If the fluid level is placed under automatic control, the computer controller receives a signal from a level detector (not shown) and adjusts the flow of liquid into and out of the collector by controlling ports 240, 245, and/or 260. As described in flow diagrams 4(a) and 4(b), the controller computer receives the an input from the level detector and adds or releases fluid to the collector if the level exceeds or falls below maximum or minimum threshold values. Alternatively, the liquid level in the collector may be maintained manually or by using one or more level detectors and a feedback circuit to adjust the flow of fluid into the collector without the use of a processor or computer.

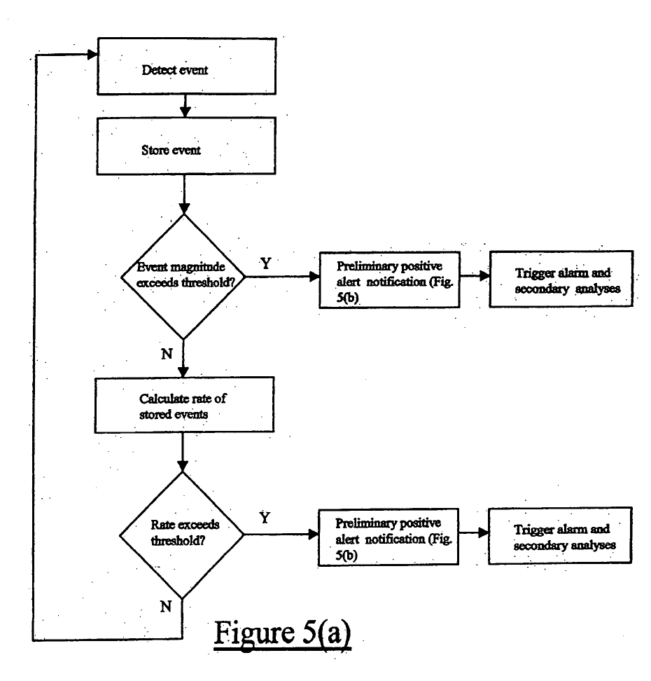

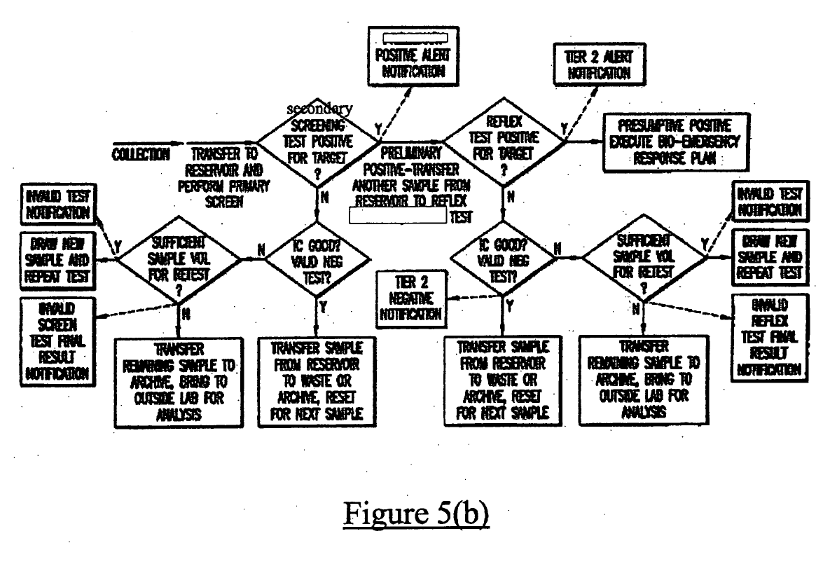

The BioCADS 10 continuously collects contaminants from the air inlet 28. The concentration of selected contaminants in the collection chamber is preferably continuously measured by the integrated detector 23, and spikes in the measured concentration of particles in the concentrator processed by the processor 20 are reported to the command and control system. In accordance with FIG. 5(a), processor 20 receives data corresponding to detection events, stores the events, and compares the detected level to a threshold value. In the event that the threshold value is exceeded, a preliminary positive alert notification is made, an alarm is triggered, and secondary analyses may be performed. In addition to monitoring and responding to the detection events themselves, whose magnitude will typically be proportional to the cumulative amount of contaminant absorbed in solution, the processor 20 also calculates the rate of change of stored events. If the rate shows a dramatic increase or spike in concentration, then a preliminary positive alert notification is also made, an alarm is triggered, and secondary analyses may be performed.

Periodically, the liquid sample containing the particles will be analyzed using bio-identifier apparatus 24, such as an automated PCR device, or alternatively a microarray, for example a microfluidic microarray biochip. This initial analysis is termed a secondary screening test. If the integrated detector 23 detects no spikes in concentration and the secondary screening test is negative for agents of interest, no action is necessary, and the facility operations will continue as usual.

If the result of in-line analysis and the secondary screening test is a “preliminary positive”, the system may be programmed to automatically perform a confirmation (Reflex)-test, optionally utilizing a criteria that is independent from the initial screening test. Preliminary positive and confirmation test results may be reported to a Visibility/Incident Response network. The results can be used to make the most appropriate decisions regarding personnel evacuation and emergency response scenarios, and further analysis of the archived sample using an outside laboratory. FIG. 5(b) is illustrative of this sequence of events.

### System Details

Site Control

Considering BioCADS disclosed herein in greater detail, the site command and control system 14 (FIG. 1) provides coordination and communication of the components in BioCADS. The command and control system 14 is preferably designed to: (a) provide a single user interface to the entire bio-detection system; (b) allow the user to quickly determine the status of ail components associated with the system; and (c) accept input to change parameters which allow for the configuration changes. At its most basic level, the command and control system 14 may provide an alarm when a “positive” reading has been obtained from the in-line detection by the integrated detector 23, and/or downstream detection by bio-identifier apparatus 24. The system 14 includes a control computer, not shown, that provides an interface to the operators and supervisors about the status of the overall system. This computer may be furthermore networked to all sensor devices (like particle counters) and to each monitor unit 12 where a plurality of monitor units are located at a particular site. The system 14 provides the higher level data collection of statistics of each component that is necessary for reports and on screen visibility. The system 14 also provides data about the test results from the bio-identifier 24.

Machine Control

The monitor unit 12 also contains a machine control processor 20 that sends and receives commands to and from the control computer of site command and control system 14. The control processor 20 performs machine control functions which: (a) controls the fluid interface between the collector/concentrator sub-system 22, the integrated detector 23, and optionally with bio-identifier apparatus 24; and (b) responds to any faults or alarms therefrom. Machine control functionality provided by the -processor 20 has been separated from the command and control system 14 to allow for the autonomous operation of the individual monitor units, coordinated by the command and control system 14. However, it would be possible to consolidate the functionality of the control processor 20 with the functionality of the command and control system 14 under a single computer controller or network of computer controllers.

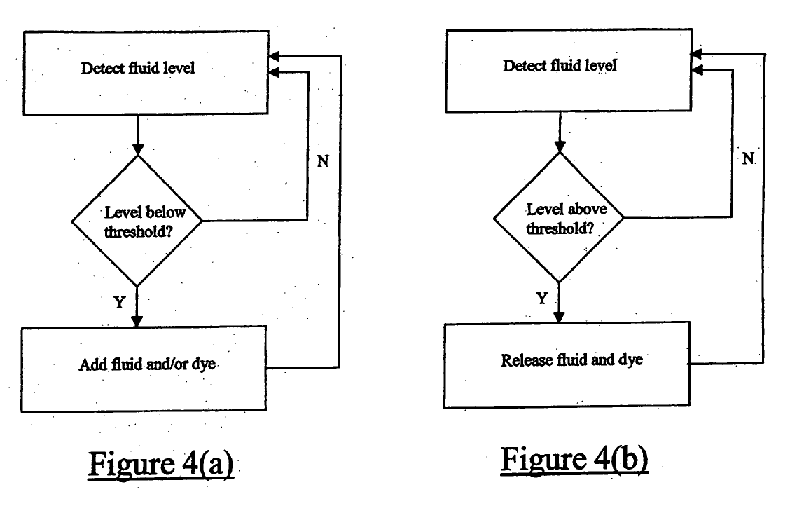

For example, as previously discussed, the machine control processor 20 may monitor and control the level of liquid-dye solution in accordance with FIG. 4(a) and FIG. 4(b), and may control the integrated detector 23 in accordance with FIG. 5(a).

Collector/Concentrator Apparatus

Several different types of collector/concentrators apparatus 22 can be used with the subject system, however, the preferred embodiment of this equipment comprises a proprietary SpinCon® system developed by Midwest Research Institute (MRI). The SpinCon® apparatus is an efficient device proven to be ideally suited for a broad range of advanced air sampling requirements, including the collection of bio-aerosols, particulate matter, and soluble vapors. The primary sample collection component of the SpinCon® system consists of a vertical glass or plastic tube, not shown, open on the top end, with a nearly tangential, vertical slit cut into the side, and is called the contactor. Fluid is placed in the contactor and air is drawn through the slit and out through the open top end of the contactor. The slit acts like a venturi/air blast atomizer; as the air passes through the slit, it speeds up and then impacts the spinning fluid in the contactor forming a wet cyclone. The collection fluid then atomizes into many small droplets, greatly increasing the surface area in contact with the air. These droplets then begin to follow the air path. The slit is only nearly tangential so the air path is across a chord of the contactor's circular cross-section. At this time, particles in the air are picked up by the fluid. As the air and droplets reach the other side of the contactor, the droplets impinge on the wall and the fluid flow is re-formed. The same fluid is re-atomized over and over, thus causing the concentration of particles in the fluid to increase linearly with time. The spinning fluid in the contactor only covers 30% to 40% of the slit, which is why only 30% to 40% of the air is sampled that is pulled into the unit.

The SpinCon® system is very effective in collecting biologicals (sizes 1-10 microns) as well as many types of smaller particles and even chemicals (agglomerated sizes<1 micron). This is due to the atomized state of the fluid at the point of collection; the massive surface area collects the larger particles, while Brownian motion, which governs the motion of small particles, enables the smaller particles to be picked up in the fluid.

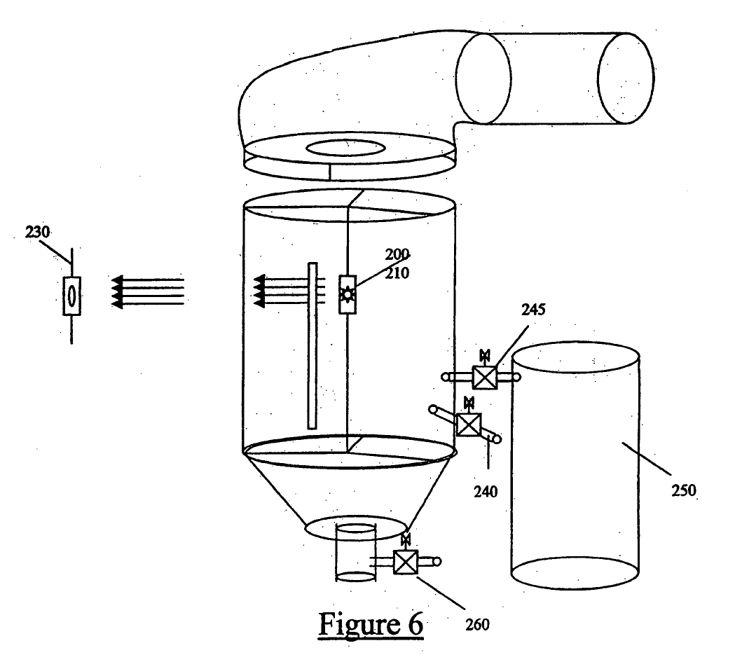

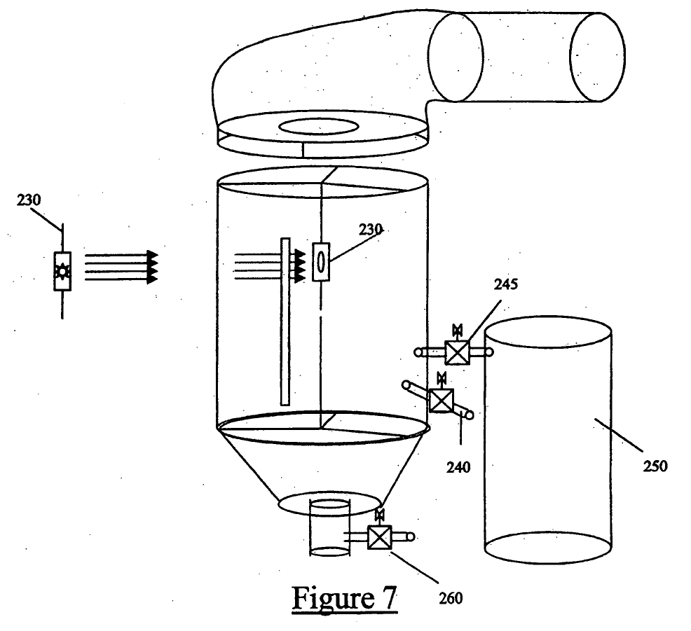

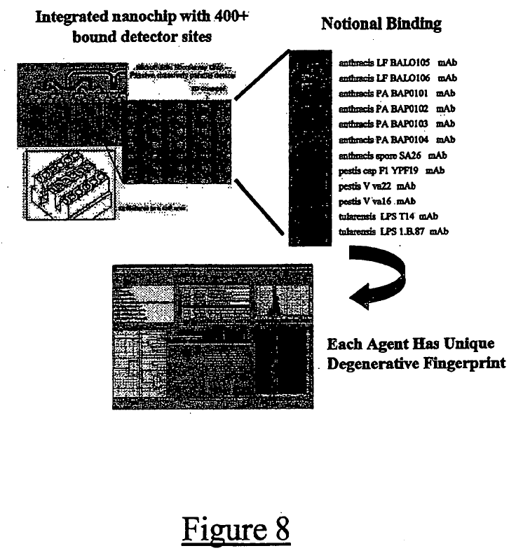

In preferred embodiments of the present disclosure the relative positions of the lamp 200, and detector 230 may vary. In one example, the transparent walls of the contactor permit light from a lamp 200 and bandpass filter 210 to pass through the walls of the contactor and interact with one or more dyes that have been introduced into the contactor. If the dyes bind to a biohazard such as a bacterial or viral pathogen, the dyes will fluoresce, creating a detectable event that may be read by a detector 230 disposed outside the contactor. Alternatively, the lamp 200 and bandpass filter 210 may be disposed inside the contactor as shown in FIG. 6, and the detector 230 is positioned outside the contactor. In yet another embodiment, the lamp 200 and bandpass filter 210 may be disposed outside the contactor as shown in FIG. 7, and the detector 230 positioned inside the contactor.

In alternative embodiments, a dry cyclonic detector, impactor, or filter system may be used to extract the contaminants from the air or other gas sample. In such systems, a liquid containing one or more dyes selected to fluoresce in the presence of targeted contaminants may be added to the particles collected in order to permit detection by a fluorescent detector, and/or prepare the sample for analysis using bio-identifier apparatus 24, such as a microfluidic microarray detector or a PCR detector.

Biohazard Dyes

In preferred embodiments of the present disclosure, one or more dyes may be used in the collector/concentrator 22 of BioCADS to identify potential biohazards, such as bacterial or viral pathogens. These dyes allow an integrated detector 23 to detect increases in the presence of a potential biohazard in a monitored area. In general, the dye is combined with a liquid sample containing nucleic acids, and incubated for a sufficient time to obtain a detectable fluorescent response. In one embodiment, one or more dyes are added to the liquid used in a liquid-assisted concentrator to allow detection of an increase in the presence of a biohazard during real-time collection of particles. The dye may be added to the liquid before it is introduced into the liquid-assisted concentrator, or alternatively may be injected into liquid already present in the concentrator so that a steady concentration of dye is present in the liquid. Alternatively, a dry concentrator may be used to isolated particles, which may then be suspended in a liquid containing one or more dyes for analysis.

Dyes that may be used in the present disclosure include, but are not limited to, calcein AM; ethidium homodimers; acridine homodimers; acridine-ethidium heterodimer; propidium iodide; hexidium iodide; 7-hydropyridocarbazoles; fluorescein; carboxy fluorescein diacetate; unsymmetrical cyanine dyes (U.S. Pat. Nos. 4,554,546 and 5,057,413, each incorporated herein by reference), such as Thiazole Orange™ (U.S. Pat. Nos. 4,883,867, 4,957,870, 5,321,130, and 5,656,449, each incorporated herein by reference) and SYBR® stains (U.S. Pat. Nos. 5,436,134, 5,534,416, 5,545,535, 5,658,751, 5,863,753, and 6,664,047, each incorporated herein by reference) (Molecular Probes, Eugene Oreg.), dimers of unsymmetrical cyanine dyes (U.S. Pat. Nos. 5,410,030 and 5,582,977, each incorporated herein by reference); acridine orange; derivatives or analogs of 5-substituted-3,8-diamino-6-phenylphenanthridium (5-DAPP); ethidium bromide; ethidium monoazide; phenanthridium dyes (U.S. Pat. No. 5,437,980, incorporated herein by reference); DAPI; Hoescht (bisbenzimide) dyes such as Hoescht 33258 and Hoescht 33342; dihydroethidium; and YO-PROe-1 and YO-YO®1 stains, SYTOX® stains, PICOGREEN® stains, and SYTO® 9 stain (Molecular Probes, Eugene Oreg.). In other embodiments, a dye used in the present disclosure may selectively bind to the surface of a bacterium, for example a protein specific for cell wall, cell envelope, or flagellum components, such as an antibody or a lectin such as wheat germ agglutinin (U.S. Pat. Nos. 5,137,810 and 5,545,535, each incorporated herein by reference).

The PICOGREEN® reagent is an ultrasensitive fluorescent nucleic acid stain that can be used to quantitate double-stranded DNA (dsDNA) in solution. Thus, this reagent can be used in BioCADS to identify increases in the presence of dsDNA, for example, from the presence of a bacterial pathogen. The PICOGREEN® assay also has a linear detection range in a standard fluorometer that extends over more than four orders of magnitude in DNA concentration with a single dye concentration. Protocols have also been developed using PICOGREEN® reagents that minimize the fluorescence contribution of RNA and single-stranded DNA (ssDNA).

The dyes used in the present disclosure may identify a range of bacterial species, whether Gram negative or Gram positive, including, but not limited to, Agrobacterium tumefaciens, Bacillus cereus, Bacillus subtilus, Clostridium sporogenes, Clostridium perfringens, Corynebacterium xerosis, Edwardsiella ictaluri, Eurioplasma eurilytica, Lactobacillus sp., Micoplasma hominus, Micrococcus luteus, Mycobacterium phlei, Propionibacterium freunderreichii, Staphylococcus aureus, Streptococcus pyogenes, Lactobacillus acidophilus, Cytophaga psychrophila, Enterobacter aerogenes, Escherichia coli, Flavobacterium meningosepticum, Klebsiella pneumonia, Neisseria subflava, Propionibacterium sp., Proteus mirabilis, Pseudomonas aeruginosa, Pseudomonas syringae, Rhizobium trifolii, Salmonella oranienburg, Serratia marcescens, Shigella sonnei, Vibrio parahaemolyticus, Zymomonas sp., or combinations thereof. In preferred embodiments, the dyes are used to detect the possible presence of bacterial pathogens, including, but not limited to, Bacillus anthracis (anthrax), Yersinia pestis (pneumonic plague), Franciscella tularensis (tularemia), Brucella suis, Brucella abortus, Brucella melitensis (undulant fever), Burkholderia mallei (glanders), Burkholderia pseudomalleii (melioidosis), Salmonella typhi (typhoid fever), Rickettsia typhii (epidemic typhus), Rickettsia prowasekii (endemic typhus) and Coxiella burnetii (Q fever), Rhodobacter capsulatus, Chlamydia pneumoniae, Escherichia coli, Shigella dysenteriae, Shigella flexneri, Bacillus cereus, Clostridium botulinum A toxoid (BoToxA), Coxiella burnetti, Pseudomonas aeruginosa, Legionella pneumophila, Staphylococcus enterotoxin B, and Vibrio cholerae.

Dyes used in the present disclosure may be first dissolved in an aqueous solution that it biologically compatible with the sample. For example, the dye may be dissolved in an aqueous solvent such as water, a buffered solution such as phosphate buffered saline (PBS), TE (10 nM Tris-HCl, 1 mM EDTA), TAE, or TBE, or a fairly polar water miscible solvent, such as dimethyl sulfoxide (DMSO), dimethyl formamide (DMF), a lower alcohol such as ethanol or methanol, or acetonitrile, to form a stock solution. Stock solutions should be protected from light prior to use. Stock solutions are then used to prepare working solutions that contain an effective amount of one or more dyes, for example by diluting the stock solution in a buffered solution, such as PBS, TE, TAE, or TBE buffers. An effective amount of dye is an amount sufficient to give a detectable fluorescent response when in the presence of nucleic acids. It is understood in the art that the specific concentration of the dyes used in the integrated detector system 23 can be routinely determined by the nature of the dyes and analysis being performed. The pH of the working solutions will be adjusted to maximize detection. When the dye in the stock solution is sensitive to moisture and hydrolyses slowly in aqueous solution, the working solution should be prepared shortly before use. Consideration of whether the dye will bind to glass will determine the material used in the collector. For example, if a dye readily binds to glass in BioCADS, plastics, such as polypropylene plastics, are preferably used in the system.

Detection of dyes binding to potential biohazards is performed by illumination at suitable wavelengths such that the presence of one or more potential biohazards are analyzed according to the fluorescent response to the illumination. Fluorescent response to illumination may be observed with any of a number of means for detecting a fluorescent response, including but not limited to visual inspection, cameras and film or other imaging equipment, or use of instrumentation such as fluorometers, plate readers, laser scanners, microscopes, or flow cytometers, or by means for amplifying the signal such as a photomultiplier.

Optimal fluorescence measurements with one or more dyes are obtained when the reagent concentrations are adjusted for the particular BioCADS system. Optimization experiments can be performed with BioCADS using methods well known to those of skill in the art to determine dye concentrations, dye combinations, times necessary for optimal fluorescence, optimal temperatures, filters, and the like Each dye used in a BioCADS should be optimized separately, and if more than one dye is used in the system, the dyes should again be optimized in combination. Preferably, reagent concentrations are selected that permit a clear distinction between live and dead cells, and/or Gram-positive or Gram-negative bacteria. Selection of filter sets, instrument sensitivity settings, and the existence or operating parameters of dry cyclone particle separator 34 (FIG. 1) may also affect the optimization. All conditions should minimize the levels of background fluorescence of the dyes prior to use in the concentrator. When more than one dye is used in BioCADS, concentration of the reagents are chosen so as to give sufficient signal to noise over the background and so that the two color intensities are reasonably matched. Preferably, the emission spectra of the two dyes are sufficiently different to permit clear visible or spectrofluorimetric separation of the individual emissions.

In certain embodiments of the present disclosure, different colored fluorescent dyes can be used to simultaneously detect live and dead bacterial cells and/or Gram-positive or Gram-negative bacteria, as generally described by Haugland, Handbook of Fluorescent Probes and Research Chemicals, Set 24 (1989-91), incorporated herein by reference. The advantage of such a system is, for example, that identification of a spike in live bacteria may indicate a greater biohazard than a sudden spike in dead bacteria, which may not be pathogenic. On the other hand, a sudden increase in the percentage of dead cells identified by BioCADS may indicate the presence of a biohazardous product in the monitored environment. Two-color fluorescent dyes have been identified that can assay bacterial viability for a diverse array of bacterial genera. Thus, the present disclosure includes methods and composition for using different colored fluorescent dyes to detect live and dead bacterial cells and/or Gram-positive or Gram-negative bacteria in a collector/concentrator apparatus.

For example, the use of two fluorogenic reagents, calcein AM and ethidium homodimer, allows for simultaneous detection of live and dead bacterial cells, as disclosed in U.S. Pat. No. 5,314,805, incorporated herein by reference. Live cells are distinguished by an intense uniform green fluorescence generated by the enzymatic hydrolysis of calcein AM. Calcein AM itself is membrane permeable and virtually non-fluorescent, which means that there is little background associated with this dye. After calcein AM is introduced into a live cell, it is hydrolysed by intracellular esterase activity to yield an intensely fluorescent product, calcein, which is retained well in live cells. Dead or damaged cells, i.e., those whose membrane integrity has been damaged, are distinguished by a bright red fluorescence resulting from nucleic acids stained with ethidium homodimer. Ethidium homodimer is excluded from live cells, thereby allowing these dyes to reliably and quantitatively distinguish between live and dead bacteria in minutes, even in a mixed population containing a range of bacterial types. The fluorescence of ethidium homodimer is enhanced approximately 40-fold after binding to nucleic acids. An advantage of this two reagent system is that either calcein AM or ethidium homodimer, but not both, can be incorporate in a single cell.

The fluorescent dyes used in LIVE/DEAD® BACLIGHT™ Bacterial Viability Kits (Molecular Probes, Eugene, Oreg.) are also useful in BioCADS. These kits utilize mixtures of the SYTO® 9 green-fluorescent nucleic acid stain and propidium iodide, a red-fluorescent nucleic acid stain. These stains differ both in their spectral characteristics and in their ability to penetrate healthy bacterial cells. When used alone, SYTO® 9 stain generally labels all bacteria in a population, both with intact membranes and damaged membranes. In contrast, propidium iodide penetrates only bacteria with damaged membranes, causing a reduction in SYTO® 9 stain fluorescence when both dyes are present. The cell type and the Gram character of the cell influence the amount of red-fluorescent staining exhibited by dead bacteria. The excitation/emission maxima for these dyes are about 480/500 nm for SYTO® 9 stain and 490/635 nm for propidium iodide, with virtually no background fluorescence. Use of these dyes can also be optimized in BioCADS so that optimal staining of bacteria can be achieved under a variety of environmental conditions.

In another embodiment, two or more dyes are used to distinguish between Gram-positive and Gram-negative bacteria. While these dyes may not distinguish between live and dead bacteria, they are able to distinguish between Gram-positive and Gram-negative bacteria. For example, the fluorescent dyes used in LIVE® BACLIGHT™ Bacterial Gram Stain Kits (Molecular Probes, Eugene, Oreg.) are useful in BioCADS. These kits utilize mixtures of the SYTO® 9 stain and hexidium iodide, a red-fluorescent nucleic acid stain (U.S. Pat. No. 5,545,535, incorporated herein by reference), to identify bacteria based on whether they are Gram-positive or Gram-negative. The SYTO® 9 stain labels both live Gram-negative and Gram-positive bacteria, whereas hexidium iodide preferentially labels live Gram-positive bacteria. In Gram-positive bacteria exposed to both dyes, the hexidium iodide stain effectively displaces the SYTO® 9 stain. The excitation/emission maxima for these dyes are about 480/500 nm for SYTO® 9 stain and 480/625 nm for hexidium iodide. Thus, when a mixed population of live Gram-negative and Gram-positive bacteria is stained with this mixture of dyes, the Gram-negative bacteria fluoresce green and the Gram-positive bacteria fluoresce red. Dead bacteria present in the system may stain variably.

BioThreat Observation and Learning Sensor (BOLS)

The integrated detector 23 of BioCADS is useful for identifying in real-time the presence of a potential biohazard, thus signaling to the appropriate personnel the need to further evaluate the potential threat. But to assess whether there is an actual threat, the potential biohazard must be identified. Therefore, in certain embodiments the system of the present disclosure will further comprise a bio-identifier apparatus 24, for example a BioThreat Observation and Learning Sensor (BOLS). A suitable BOLS comprises a chemical/biochemical reaction system that will perform high-throughput assays to determine whether the biological agent identified is a potential biohazard, as well as the identity of the actual biohazard. Preferably, BOLS will have chemical fluidic vessels for parallel performance of pluralities of chemical reactions, for example in a microfluidic microarray chip. This system will also preferably have an integrated nanochip that uses pattern recognition analysis based on the reactions that occur in the chip to identify the biological threat. The pattern recognition analysis may also further elucidate the relative pathogenicity of the identified biohazard. For example, BOLS can be programmed to recognize a fingerprint binding pattern that is unique to various biohazards, such as bacterial or viral pathogens, or other toxins, by leveraging the varying degrees of specificity and non-specificity between the potential biohazard and a large panel of antigens, for example nucleic acids, oligonucleotides, peptides, proteins, antibodies, oligosacchrides, phospholipids, and other biopolymers. The pattern recognition analysis can be performed by processor 20. Alternatively, this and other functions of the BOLS control software can operate as a dedicated processor or the site command and control system 14 (FIG. 1).

BOLS has many advantages for rapidly identifying a potential biohazard. For example, BOLS can utilize a nanoliter environment, which means that only small amounts of reagents are needed for analyzing potential biohazards, and binding events are more likely to utilize first order kinetics, which will result in more rapid reactions. The sample will also pass through a microfluidic microarray chip rapidly, and can be recirculated through the chip as necessary. In addition, it will be difficult to modify known bacterial or viral pathogens to avoid detection by BOLS because it will use multiple detection points. The algorithm thresholds used by BOLS can be adjusted to minimize false positive or false negative results, and can also be synchronized with alarms and wireless reporting. Also, BOLS will preferably include a reusable microfluidic microarray chip which can be flushed with buffer between uses, and will take up minimal space.

In certain embodiments, BOLS can also be trained to identify new threats with no adjustments to the hardware or microarray chips by passing newly identified biohazards over the chip and identifying the unique binding pattern for the biohazard on the chip. The BOLS software can then be updated to correlate the new binding pattern with a particular biohazard on the existing system. Another advantage of BOLS is that all fluidic activity for detection occurs automatically and is completely contained inside the system, thereby minimizing the risk of inadvertent contamination of the environment or instrument with the identified biohazard. In addition, samples can be retained for further analysis.

In certain embodiments, BioCADS automatically loads a liquid sample form the collector/concentrator 22 into BOLS, for example a microfluidic microarray chip. Preferably, any extraneous chemicals or other components that may impede the detection reactions within the chip, such as dirt, will be removed prior to introducing the fluid sample into the chip. The fluid sample is automatically transported through the chip and exposed to various panels of bound capture molecules, such as antigens, nucleic acids, oligonucleotides, peptides, proteins, antibodies, oligosacchrides, phospholipids, and other biopolymers, in discrete locations or vessels. The bound capture molecules can either be synthesized in the chip or presynthesized and attached at discrete predetermined locations in the chip. Preferably, BOLS of the present disclosure are able to perform a large number of parallel chemical reactions without the use of a large number of valves, pumps, or other complicated fluidic components. In some embodiments, the particles in the sample may be exposed to conditions that will cause any bioparticles to lyse, for example by exposing the bioparticles to certain chemicals or ultrasound, thereby releasing the DNA from inside the bioparticles, which may then bind to specific oligonucleotides in the chip to determine the identity of the biological agent.

In particular embodiments of the present disclosure, BOLS utilizes the flow-through technology disclosed in U.S Publication No. 2002-0012616, incorporated herein by reference. This application broadly discloses a microarray biochip for parallel microfluidic reactions. The technology can also be used to synthesize desired bound capture molecules using in situ parallel combinatorial synthesis with photogenerated reagents at predetermined reaction sites in the chip. These biochips are straight-forward to handle because the bound capture molecules are located in the chip, not on the surface, which allows for a microfluidic system to be used to analyze samples isolated by BioCADS. The biochips have small internal volumes, which provide a highly efficient molecular contacting environment. In addition, these biochips are potentially reusable. The biochip can also be designed to minimize or prevent the intermixing of active reagents or samples between discrete reaction cells as long as certain fluid flow conditions are maintained.

For example, the microfluidic array chip can be a (external) pressure driven device containing channels which are arranged such that reagents or samples are distributed to discrete reaction cells. In predetermined reaction cells reactive chemical reagents are generated in situ by light exposure from an external light source, such as a digital micromirror device (DMD). The chip itself can be miniaturized. An exemplary chip measures approximately 1.5×2.0×0.1 cm, contains up to approximately 27,000 discrete reaction cells, and has a total internal volume of only 10 μl. Within the chip, the cross-section dimensions of the fluid channels and reaction cells are very small (on the order of tens of microns), and the mass transfer between the surface and the liquid is significantly enhanced as compared to larger sized reactors. This design significantly enhances the rate of chemical reactions within the chip.

In-depth analysis of multiple arrays, samples, and hybridization events to identify patterns and threshold algorithms based on reactions that occur on the chip may be performed using a variety of commercially available software packages, such as Vector Xpression 3.1 or Vector PathBlazer 2.0 (Invitrogen, USA). The BOLS software will allow complementation patterns on the chips to be correlated with specific biological agents. The software utilized by 84LS will preferably provide database storage and handling, cluster validation, annotation, and import of data files, as well as the ability to perform numerous data and statistical analyses including: hierarchical (e.g., linkage analysis) and non-hierarchical (e.g., self-organizing maps) methods, distance/similarity options, and 2-group and 3-group methods (e.g., one-way ANOVA).

An embodiment of BOLS is shown in FIG. 9. In brief summary, after triggering and collection, a small aliquot of collected material (e.g., 25 μl) is filtered and passed into a nanochip with etched 100 gm channels. Each channel contains panels of bound capture molecules (including antibodies and protein receptors), wherein the bound capture molecules are printed or synthesized in situ in the channels. The bound capture molecules form an addressable surface because the location of each capture molecule is known. Particle in the liquid sample passes over each address under conditions (e.g., flow-rates and pH) that allow binding. Based on the kinetic binding characteristics between the population of particles and the antigens present in the chip, the biological particles (e.g., bacteria, virus, or other toxin) have the opportunity to adhere to each capture site. In most cases, the particles will bind to multiple capture sites. The concentration of particles in each capture site, however, will vary according to the identify of the biological particles. This variance provides a pattern that is unique for each biohazard. Examples of two different visualization techniques that can be used to identify the binding pattern are interference of light caused by the captured organism (e.g., darkfield microscopy), or the use of a robust and general rapid dye (e.g., PICOGREEN) that enhances visualization and increases sensitivity.

System Operation

The operation of BioCADS as disclosed herein is controlled by the machine control processor 20, and its operation is synchronized with other equipment by the command and control system 14. The flow chart shown in FIG. 5(b) is illustrative of the operational sequence.

Prior to collecting samples, BioCADS must be initialized and prepared for data collection. The following describes the tasks involved: (1) start-up of site command and control system; (2) set collection parameters. The collection parameters include the setup for each run in sequential order of steps. Preferably, the run setup will indicate the machine ID sample number, start time, stop time, and the assay description. The command sequences are stored locally in the machine control processor 20 (FIG. 1). The supervisory system 14 will have the capability to download a new assay description and associated command sequence to the machine control processor.

At the specified start time, BioCADS will initiate the air collection process. This enables the collector/concentrator sub-system 22 to start operation. An indicator 25 on the cabinet 26 (FIG. 3) provides an indication that the system is active. Air is then sampled from the air intake or manifold 28 where it may be routed via tube 32 which is a grounded anti-static tube to the dry cyclone pre-separator 34 that is designed to eliminate particles that are larger than the inhalation threat range of 1-10 microns. From the dry-cyclone 34, the sampled aerosol may be routed to a collector/concentrator 22, for example a SpinCon®, which, as noted above, impinges the air into a small volume of liquid. The liquid assisted cyclonic collector may operate at a flow of about 450 lpm. As air passes through the unit, cyclonic mixing transfers a high portion of the target particles into the liquid. The liquid medium remains in the collector/concentrator 22 to continuously concentrate the target particles into the liquid. At the start of the collection process, liquid with a suitable concentration of one or more dyes is injected into the system. The dyes may be added to the liquid either before or after injection into the system. During the collection, the liquid level is monitored, and evaporated liquid is replaced by injecting makeup liquid to maintain the appropriate sample volume.

As the cyclonic collector extracts particles from air or other gas, the particles are concentrated in the liquid solution containing one or more dyes. The dye binds to nucleic acids or other markers of selected bioactive agents that are captured in the liquid, and fluoresces in response to a light source that provides incident light through the walls of the contact chamber. The processor 20 responds to signals generated by the detector 230 (FIG. 3). If either the concentration or the rate of change in concentration exceeds the corresponding threshold values, the processor will generate an alert which may trigger an alarm and/or additional analyses, for example by the bio-identifier apparatus 24.

At a planned “stop time” or in response to a trigger input, the machine control processor 20 sends a signal to the collector/concentrator 22 to transfer a liquid sample out for analysis. The collection process is paused while the sample is transferred into one or more bottles or tubes of a collection reservoir, and the collector/concentrator 22 is then refilled to start the next collection window. In the event that the downstream detector 24 is of a continuous flow variety, the processor 20 continuously permits fluid to exit the contact chamber of the collector/concentrator 22 at a controlled rate while maintaining the proper fill level in the contact chamber by operating the control valves to replenish the supply of water and dye in accordance with FIG. 4(a) and FIG. 4(b).

What is claimed is:

1. A system for detecting a biological agent, comprising:
a) a collector/concentrator apparatus for collecting a sample of particles from a gas at a monitored location, wherein the sample of particles is suspended in a liquid comprising one or more dyes that detect the presence of nucleic acids; and
b) an integrated detector comprising a light source and a light detector, wherein the integrated detector can detect a change in the fluorescence of the liquid, wherein increased fluorescence of the liquid indicates the presence of a biological agent in the sample.

2. The system of claim 1, wherein the biological agent is a bacterial pathogen.

3. The system of claim 1, wherein the collector/concentrator apparatus is a liquid-assisted collector.

4. The system of claim 3, wherein the liquid-assisted collector is a SpinCon® apparatus.

5. The system of claim 1, wherein the collector/concentrator apparatus operates continuously to collect samples of particles.

6. The system of claim 1, wherein the light source is a lamp.

7. The system of claim 6, wherein the lamp is a White Arc lamp.

8. The system of claim 1, wherein the light detector is an optical detector.

9. The system of claim 1, wherein the light detector is a photomultiplier tube.

10. The system of claim 1, wherein the light detector is a fluorometer.

11. The system of claim 1, wherein the light source is inside the collector/concentrator apparatus.

12. The system of claim 1, wherein the light source is outside the collector/concentrator apparatus.

13. The system of claim 1, wherein the light detector is inside the collector/concentrator apparatus.

14. The system of claim 1, wherein the light detector is outside the collector/concentrator apparatus.

15. The system of claim 1, wherein the light source and the light detector are adjacent to the collector/concentrator apparatus.

16. The system of claim 1, wherein light from the light source traverses at least one wall of the collector/concentrator apparatus.

17. The system of claim 1, wherein the integrated detector further comprises a bandpass filter.

18. The system of claim 1, wherein the integrated detector comprises a plurality of light sources.

19. The system of claim 1, wherein the integrated detector comprises a plurality of light detectors.

20. The system of claim 1, wherein the system further comprises a dry-cyclone particle separator.

21. The system of claim 1, wherein the system further comprises a particle counter.

22. The system of claim 1, wherein the system further comprises a bio-identifier apparatus.

23. The system of claim 22, wherein the bio-identifier apparatus comprises a polymerase chain reaction (PCR) biological agent identifier.

24. The system of claim 22, wherein the bio-identifier apparatus comprises a microfluidic microarray biochip.

25. A method of detecting a biological agent, comprising:
a) collecting a sample of particles from a gas at a monitored location using a liquid-assisted collector, wherein the liquid-assisted collector comprises a liquid with one or more dyes that detect the presence of nucleic acids;
b) exposing the sample to a light source; and
c) detecting the fluorescence of the liquid in the sample with a light detector;
wherein increased fluorescence of the liquid in the sample indicates the presence of a biological agent.

26. The method of claim 25, wherein the biological agent is a bacterial pathogen.

27. The method of claim 25, wherein the liquid-assisted collector is a SpinCon® apparatus.

28. The method of claim 25, wherein the dyes are selected from the group consisting of PICOGREEN®, calcein AM, ethidium homodimer, SYTO® 9, propidium iodide, and hexidium iodide.

29. The method of claim 26, wherein the increased fluorescence indicates the presence of a live bacterial pathogen.

30. The system of claim 26, wherein the increased fluorescence indicates the presence of a dead bacterial pathogen.

31. The system of claim 26, wherein the increased fluorescence indicates the presence of a Gram-positive bacterial pathogen.

32. The system of claim 26, wherein the increased fluorescence indicates the presence of a Gram-negative bacterial pathogen.

33. The method of claim 25, wherein the light source is a lamp.

34. The method of claim 33, wherein the lamp is a White Arc lamp.

35. The method of claim 25, wherein the light detector is an optical detector.

36. The method of claim 35, wherein the optical detector is a photomultiplier tube.

37. The method of claim 25, wherein the sample is transferred to a bio-identifier apparatus to determine the identity of the biological agent.

38. The method of claim 37, wherein the bio-identifier apparatus comprises a polymerase chain reaction (PCR) biological agent identifier.

39. The method of claim 25, wherein the bio-identifier apparatus comprises a microfluidic microarray biochip.
